(19) United States
(12) Patent Application Publication
(10) Pub. No.: US 2010/0028286 A1
(43) Pub. Date: Feb. 4, 2010

(54) Method and Composition for Stabilizing Hair Moisture Levels

(76) Inventors: Jose Antonio Carballada, Cincinnati, OH (US); Timothy Ray Nijakowski, Mason, OH (US); Bryan Patrick Murphy, Loveland, OH (US); Alexis M.J.A. Huyghues-Despointes, Villa Angla (VE); Alex Kalbfleisch, Darmstadt (DE)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/507,221

(22) Filed: Jul. 22, 2009

Related U.S. Application Data
(60) Provisional application No. 61/085,238, filed on Jul. 31, 2008.

(51) Int. Cl. A61K 8/81; A61Q 5/12

(52) U.S. Cl. 424/70.16

(57) ABSTRACT

A method and composition for maintaining and/or stabilizing moisture levels in the hair shaft comprising the application of a hair care composition comprising a copolymer comprising at least one hydrophilic monomer and at least one hydrophobic monomer, and a dermatologically acceptable carrier wherein the hydrophilic and hydrophobic monomers undergo polymerization inside the hair fibers.

### CROSS REFERENCE TO RELATED APPLICATION

This application claims the benefit of US Provisional Application No. 61/085,238, filed on Jul. 31, 2008.

### FIELD OF THE INVENTION

The present invention relates to a method and composition for stabilizing moisture levels in the hair via a method for application of a hair care composition comprising at least one hydrophilic monomer and at least one hydrophobic monomer that copolymerize inside the hair fiber.

### BACKGROUND OF THE INVENTION

Human hair properties are greatly affected by water. Dry, arid conditions can leave hair in a dry, rough, lusterless, or frizzy condition due to removal of the hair's internal moisture via acclimation to surrounding environmental conditions. In addition, hair may possess increased levels of static after drying, which can interfere with combing and reduce hair manageability. This results in a condition commonly referred to as “flyaway hair.” Certain consumers consider such flyaway hair and the corresponding increase in total hair volume undesirable. The opposite extreme moisture level can also lead to undesirable hair conditions. A wet, humid environment can cause an increase in the moisture level of hair. Hair that absorbs too much moisture can be limp, and flat, failing to hold a desired style.

A variety of approaches have been developed to address these issues. Such approaches typically seek to either increase or decrease the moisture level in the hair utilizing a variety of hair care actives. However, these approaches only serve to fix a problem after it already exists.

Accordingly, the need remains for a hair care composition and a method of using the composition, which stabilizes, or maintains a certain moisture level in the hair, preventing the hair from either i) becoming too dry when moisture leaves the hair because the environmental conditions are arid, or ii) absorbing too much moisture when the conditions in the environment are humid.

### SUMMARY OF THE INVENTION

A method for maintaining moisture in the hair shaft comprising the steps of chemically modifying the internal region of a hair shaft by applying to hair a composition comprising a copolymer comprising at least one hydrophilic monomer and at least one hydrophobic monomer, wherein the at least one hydrophilic monomer is selected from the group consisting of sulfopropyl(meth)acrylate compound 2-acrylamidodiglycolic acid, hydroxymethyl diacetone acrylamide, (3-acrylamidopropyl)trimethyl ammonium salt, 2-aminoethyl methacrylate and mixtures thereof; and wherein the at least one hydrophobic monomer is selected from the group consisting of 2-ethoxyethyl acrylate, 2-butoxyethyl acrylate, alpha methylene lactone and mixtures thereof and a dermatologically acceptable carrier.

A hair care composition comprising a copolymer comprising from about 20% to about 99% hydrophilic monomer, wherein the hydrophilic monomer is hydroxymethyl diacetone acrylamide, and from about 1% to about 80% of a hydrophobic monomer, wherein the monomer is butoxyethylacrylate; and a dermatologically acceptable carrier.

### BRIEF DESCRIPTION OF THE DRAWINGS

While the specification concludes with claims which particularly point out and distinctly claim the present invention, it is believed that the present invention will be better understood from the following description of preferred embodiments, taken in conjunction with the accompanying drawings, in which like reference numerals identify identical elements and wherein:

FIG. 1 is a line graph of acceptable hair moisture levels;

FIG. 2 is a graph showing the percentage of water absorbed by the hair after treatment with the copolymer.

### DETAILED DESCRIPTION OF THE INVENTION

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

“Hair,” as used herein, means hair on the human head and scalp. “Hair shaft” means an individual hair, and may be used interchangeably with the term “hair.”

“Internal region of the hair shaft,” as used herein, means any non-surface portion of the hair shaft, including the inner portion of the cuticle. “Non-surface portion” may be understood to mean that portion of the hair that is not in direct contact with the outside environment.

“Proximal to the scalp,” as used herein, means that portion of an extended, or substantially straightened, hair shaft that is closer in distance to the scalp than to the end of the hair. Thus, about 50% of the hair would be considered proximal to the scalp, and about 50% of the hair would be distal to the scalp. “x cm proximal to the scalp” means a distance “x” along the hair, with one endpoint being on or directly adjacent to the scalp, and the second endpoint being measured “x” centimeters along the length of the extended or substantially straightened hair.

“Dermatologically-acceptable carrier,” as used herein, means that the compositions or components described are suitable for use in contact with human keratinous tissue without undue toxicity, incompatibility, instability, allergic response, and the like.

“Derivatives,” as used herein, includes but is not limited to, amide, ether, ester, amino, carboxyl, acetyl, and/or alcohol derivatives of a given compound.

“Monomer,” as used herein, means a discrete, non-polymerized chemical moiety capable of undergoing polymerization in the presence of an initiator.

“Ethylenic monomer,” as used herein, means a chemical species that contains an olefenic carbon-carbon double bond (C═C) and is capable of undergoing polymerization in the presence of an initiator.

“Chemically modify,” or grammatical equivalents thereof, as used herein, means that a chemical moiety such as monomer and/or crosslinker and/or polymer, stably affixes to a second chemical moiety, for example, a keratin protein, another component of hair, and/or another monomer or crosslinker.

“Stably affix” is understood to include both covalent and non-covalent forms of chemical bonds that once formed, remain unchanged through wetting washing, styling and other types of hair treatment. In general, stably affixed chemical moieties may not be removed from the hair without damaging or substantially destroying the hair.

“Moisture Stabilization” as used herein, is determined by the Thermal Gravimetric Analysis Method. One example of determining moisture stabilization is as follows: First, a 10 g switch of hair in the form of a ponytail is equilibrated for 48 hrs at various environmental conditions having different vapor densities (see chart below). The weight of the switch is recorded at each condition.

| | Temperature | | |
|---|---|---|---|
| Vapor Density (g/m³) | (° F.) | Relative Humidity (%) | Condition |
| 2.33 | 30 | 50 | Arid |
| 9.21 | 70 | 50 | Moderate |
| 17.19 | 90 | 50 | Humid |

Finally the hair is super dried by heating the switch in a drying oven at 120° C. for 4 hrs and measuring its weight. The difference in weight calculated from the gravimetric measurements at each condition and dry weight represent the amount of water the hair contained at its equilibrium which is generally between about 4-25% throughout the range of conditions. Moisture stabilization is improved if the hair is treated with the present invention if the percent of water contained in the hair through the same range of conditions is narrowed.

“Reserve alkalinity,” as used herein, means the relative strength and apparent concentration of the base used to adjust the pH, measured as described in ASTM D 1121, wherein the base of the present invention is substituted for the antifreeze used in the method.

“Reducing Composition” as used herein, means any form of product containing a reducing agent such as thioglycolate (about 0.1%-about 5% by weight) that is used to pre-treat the hair to induce greater swelling and uptake of actives.

“Finishing Composition” as used herein, means a neutralizing and/or oxidizing composition and can be any form of product containing an oxidizer such as hydrogen peroxide (about 0.1%-about 3% by weight) and buffered at low pH that is used to neutralize reduction and alkaline effects in the hair.

“Kit,” as used herein, means a packaging unit comprising a moisture stabilization composition and a separately packaged second composition, as described herein.

“Separately packaged,” as used herein, means any form of packaging that prevents a moisture stabilization composition from coming into physical contact, or admixing, with a second composition. “Separately packaged” may mean that the individual compositions are packaged in separate containers, or alternatively in a single container partitioned such that the compositions are not in physical contact.

“Implement,” as used herein, means a device used to facilitate application of a composition to the hair and/or manipulation of the hair. Examples of implements include, but are not limited to, a comb, a means for directed delivery (e.g., an applicator or tube), a covering for the hair (e.g., plastic bag, shower cap, etc.), and combinations thereof.

“Energy delivery device,” as used herein, means any device used to deliver energy to keratinous tissue, including the hair and scalp. “Delivery of energy,” means that the surface of the keratinous tissue is exposed to the energy emanating from the energy delivery device, where it may penetrate to the desired layers of the tissue, including the hair shaft and/or hair follicle. Energy includes but is not limited to energy in the form of light, heat, sound (including ultrasonic waves), electrical energy, magnetic energy, electromagnetic energy (including radiofrequency waves and microwaves), and combinations thereof.

### I. METHOD FOR STABILIZING HAIR MOISTURE LEVELS

The present invention relates to a method for maintaining and/or stabilizing moisture levels in the hair shaft comprising the steps of chemically modifying the internal region of a hair shaft by applying to hair a composition comprising at least one hydrophilic monomer and at least one hydrophobic monomer that copolymerize inside the hair fiber, wherein the at least one hydrophilic monomer is selected from the group consisting of sulfopropyl(meth)acrylate, 2-acrylamidodiglycolic acid, hydroxymethyl diacetone acrylamide, (3-acrylamidopropyl)trimethyl ammonium salt, 2-aminoethyl methacrylate and mixtures thereof, and wherein the at least one hydrophobic monomer is selected from the group consisting of 2-ethoxyethyl acrylate, 2-butoxyethyl acrylate, alpha methylene lactone, tetrahydrofurfuryl acrylate, Methyl 3-hydroxy-2-methylenebutyrate, and mixtures thereof and a dermatologically acceptable carrier. The invention also relates to the use of this composition for maintaining and/or stabilizing the moisture level in the hair.

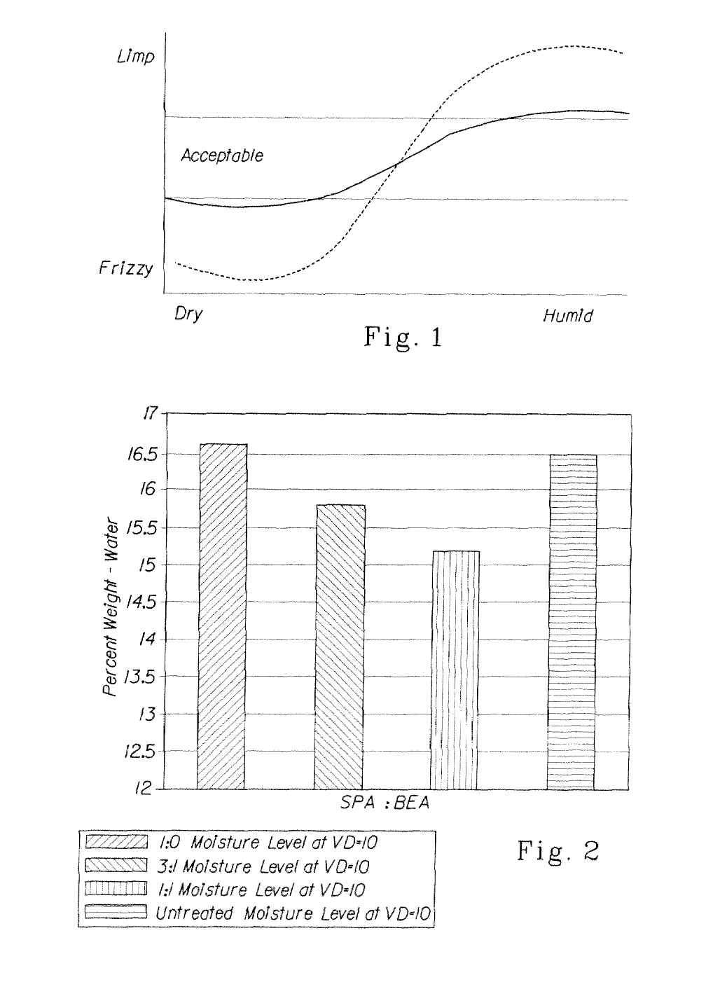

Use of this method can result in maintaining or stabilizing the moisture level within the hair shaft, thereby achieving consumer desirable hair. As shown in the line graph of FIG. 1, there is a particular range of moisture level that is acceptable by the consumer. If the moisture level is maintained within this acceptable range, the hair exhibits consumer desirable characteristics (i.e. the hair is not limp, frizzy, or overly dry). Shown in FIG. 2 is a bar graph which shows the percent weight of water in different hair switches when the hair switch is either untreated, treated with a single monomer, or treated with a copolymer. In the embodiments shown in FIG. 2 the copolymer is formed from sulfopropyl(meth)acrylate (“SPA”) and butoxyethyl acrylate (“BEA”). In the embodiment wherein the ratio of SPA to BEA is 3:1 the percent weight of water of the hair switch is about 15.75% water. In the embodiment wherein the ratio of SPA to BEA is 1:1, the percent weight of water of the hair switch is about 15.25% water. In the example wherein the SPA only was used, and no copolymer was formed, the percent weight of water of the hair switch is more than about 16.5% water. In the example where the hair was untreated the percent weight of water of the hair switch is about 16.5% water. Therefore, this bar graph indicates that when the hair is treated with the copolymer of the present invention, less water is absorbed in similar environmental conditions. This is a particularly useful treatment in humid conditions when you want to minimize the amount of water absorbed by the hair.

### II. MOISTURE STABILIZATION COMPOSITIONS

The present invention comprises a moisture stabilization composition comprising a copolymer comprising at least one hydrophilic monomer and at least one hydrophobic monomer. In one embodiment the at least one hydrophilic monomer is selected from the group consisting of sulfopropyl(meth)acrylate, 2-acrylamidodiglycolic acid, hydroxymethyl diacetone acrylamide, (3-acrylamidopropyl)trimethyl ammonium salt, 2-aminoethyl methacrylate and mixtures thereof; and the at least one hydrophobic monomer is selected from the group consisting of 2-ethoxyethyl acrylate, 2-butoxyethyl acrylate, alpha methylene lactone, tetrahydrofurfuryl acrylate, methyl 3-hydroxy-2-methylenebutyrate, and mixtures thereof and a dermatologically acceptable carrier. The ethylenic monomer is of a size suitable to penetrate the hair shaft and is soluble or dispersible in the carrier. The moisture stabilization composition may have a pH of about 7.0 and below, alternatively of from about 3.0 to about 7.0, and alternatively of from about 5.0 to about 6.0. In all embodiments, “about,” when used in reference to pH, is understood to mean±0.5 pH unit.

Depending on the desired affect (i.e. reducing moisture loss, or preventing moisture absorption) the ratio of the hydrophobic and hydrophilic monomer can be adjusted. In one embodiment the copolymer can comprise from about 20% to about 99% hydrophilic monomer, and from about 1% to about 80% hydrophobic monomer. In another embodiment the copolymer can comprise from about 25% to about 75% hydrophilic monomer, and from about 5% to about 20% hydrophobic monomer. If the conditions are wet and/or humid the copolymer can comprise from about 40% to about 60% hydrophobic monomer and about 40% to about 60% hydrophilic monomer. In another embodiment the copolymer can comprise from about 45% to about 55% hydrophobic monomer and from about 45% to about 55% hydrophilic monomer. In yet another embodiment the copolymer can comprise about 50% hydrophobic monomer and about 50% hydrophilic monomer. If the environmental conditions are dry and/or arid the copolymer can comprise from about 1% to about 10% hydrophobic monomer and from about 90% to about 99% hydrophilic monomer. In another embodiment the copolymer can comprise from about 5% to about 10% hydrophobic monomer and from about 90% to about 95% hydrophilic monomer. In yet another embodiment the copolymer can comprise about 5% hydrophobic monomer and about 95% hydrophilic monomer. It is further contemplated that a single monomer can be used for either dry or humid conditions, and another composition can be added to the hair to activate the polymer or copolymer to either retain or repel water absorption. This composition could be delivered as a shampoo or any other hair care composition, including but not limited to, a rinse and/or rinse off treatment, a leave on treatment, a tonic, a gel, a mousse etc.

In one embodiment the hydrophilic monomer is a hydroxylalkylacrylamide. “Hydroxylalkylacrylamide” as used herein, is understood to include derivatives, salts and/or isomers of hydroxylalkylacrylamide. The hydroxylalkylacrylamide compounds of the present invention are understood to be in a non-polymerized form, and do not include polymers of hydroxylalkylacrylamide. One example of a useful hydroxylalkylacrylamide compound is hydroxymethyl diacetone acrylamide, CAS #42772-51-0.

In one embodiment the hydrophobic monomer is alkoxyacrylate. “Alkoxyacrylate” as used herein, is understood to include derivatives, salts and/or isomers of alkoxyacrylate. The alkoxyacrylate compounds of the present invention are understood to be in a non-polymerized form, and do not include polymers of alkoxyacrylate. One example of a useful alkoxyacrylate compound is butoxyethylacrylate, CAS #7251-90-3.

The moisture stabilization composition further may comprise at least one ethylenic monomer having a size suitable to penetrate the hair shaft and a molecular weight of 500 g/mole or less, alternatively from about 50 g/mole to about 500 g/mole, alternatively from about 75 g/mole to about 400 g/mole, and alternatively from about 100 g/mole to about 400 g/mole. Examples of ethylenic monomers suitable for use in the moisture stabilization composition of the present invention include, but are not limited, to hydroxylalkylacrylamide, alkoxyacrylate, mesaconic acid, 2-pentenoic acid, tiglic acid, tiglic acid esters, furan-3-acrylic acid, 2-acrylamido-2-methyl-1-propanesulfonic acid, maleamic acid, 3-aminocrotonic acid, crotonic acid esters, itaconic anhydride, trimethylsilylacrylate, poly(ethyleneglycol)acrylates, N-vinylacetamide, 2-acetamidoacrylic acid, vinylsulfonic acid, tetrahydrofurfurylacrylate, N-methyl-N-vinylacetamide, vinylpropionate, vinylanisole, vinylcrotonate, methyl 3-hydroxy-2-methylenebutyrate, methacryloyl-L-lysine, N-(2-hydroxypropyl)methacrylamide, 2-acrylamidodiglycolic acid, 2-ethoxyethyl acrylate, 2-butoxyethyl acrylate, N-isopropylmethacrylamide, 2-aminoethyl methacrylate, 2-bromoethyl acrylate, 3-(dimethylamino)propyl acrylate, (3-acrylamidopropyl)trimethyl ammonium salt, [2-(acryloyloxy)ethyl]-trimethylammonium salt, and salts, isomers, derivatives and mixtures thereof. The moisture stabilization composition may comprise from about 0.1% to about 20%, alternatively from about 1% to about 15%, alternatively from about 5% to about 10%, alternatively from about 0.1% to about 10%, and alternatively from about 10% to about 20%, of an ethylenic monomer.

One method of selecting compounds for this technology is selecting from a group of ethylenic monomers, the monomer can be from the family of acrylates, acrylamides, or vinyls. The monomer can be water soluble and less than 500 amu's in molecular weight. The monomer can penetrate hair to the extent of 5-10% by weight using an aqueous carrier and gravimetric techniques to measure. The monomer may react to about 75% completion within 30 minutes at 35° C. in presence of initiator. In another embodiment the monomer may react from about 90% to about 100% completion within 30 minutes at 35° C. in presence of initiator. The final resulting polymer can be water soluble or remove-able from the surface of hair via normal shampooing. These monomers can be used alone or as co-monomers with other actives to enhance reactivity and solubility.

The moisture stabilization composition further may comprise at least one crosslinker having a molecular weight of a size suitable to penetrate the hair shaft and a molecular weight of 500 g/mole or less, alternatively from about 100 g/mole to about 500 g/mole, alternatively from about 100 g/mole to about 400 g/mole, and alternatively from about 200 g/mol to about 400 g/mole. Examples of crosslinkers suitable for use in the moisture stabilization composition of the present invention include, but are not limited to 1,4-bisacryloylpiperazine, methylenebisacrylamide, ethylenebisacrylamide, divinylbenzene, poly-ethyleneglycol di(meth)acrylate, ethylene glycol di(meth)acrylate, 1,3-butanediol di(meth)acrylate, 1,4-butanediol di(meth)acrylate, neopentyl glycol di(meth)acrylate, Bis[2-(methacryloyloxy)ethyl]phosphate, N,N′-bis(acryloyl)cystamine, N,N-Diallylacrylamide, triallyl cyanurate, 3-(Acryloyloxy)-2-hydroxypropyl methacrylate and mixtures thereof.

In one embodiment, the ratio of the weight percentage of the hydroxyalkylacrylamide compound, the alkoxyacrylate, and/or the mixture thereof to the weight percentage of the crosslinker is from about 50:1 to about 10:1, alternatively from about 40:1 to about 10:1, and alternatively from about 20:1 to about 10:1.

The present invention further may comprise, in some embodiments, a second composition comprising an initiator, useful for promoting binding of an ethylenic monomer and/or crosslinker to the keratin and/or to another monomeric compound. The second composition may comprise from about 0.001% to about 5%, alternatively from about 0.01% to about 3%, and alternatively from about 0.1% to about 1%, of an initiator. Examples of suitable classes of initiators include, but are not limited to, peroxodisulfates, peroxides, peracids, percarbonate, phosphates, manganates, borates, bis-alkyamidines, sulfites, peroxyesters, bis-cyanocarboxylic acids, alpha-amino acetic acids, and mixtures thereof. Non-limiting examples of suitable initiators include sodium peroxydisulfate, 2,2′-azobis(2-methylpropionamidine)dihydrochloride, 2,2′-azobisisobutyronitrile, benzoyl peroxide, peracetic acid, ammonium cerium(IV) nitrate, hydroxymethanesulfinic acid and mixtures thereof. The second composition further may have a pH of below 7.0, alternatively of from about 3.0 to about 7.0, alternatively the second composition may be a neat material existing not as a solution but as a powder or crystals packaged individually.

The moisture stabilization composition further may comprise from about 0.01% to about 1% of at least one organic or inorganic catalyst. Non-limiting examples of suitable organic catalysts include 2-pyrrolidinoethanol, 1-piperidine-ethanol, 4-methylmorpholine, 2-morpholinoethanol, tetramethylethylenediamine, and mixtures thereof. Non-limiting examples of suitable inorganic catalysts include salts and/or hydrates of cerium, cobalt, manganese, iron, nickel, copper, and mixtures thereof.

The present invention may comprise a third composition comprising a alkali agent, which is packaged separately from the first and the second composition, and which is useful for adjusting the pH of one, or a combination of any, of the compositions disclosed herein. The third, or pH adjusting, composition may have a pH of above 7.0, alternatively of from about 7.0 to about 12.0, alternatively of from about 8.0 to about 12.0, and alternatively of from about 9.0 to about 11.0. Additionally or alternatively, the third composition has a reserve alkalinity from about 1 to about 40, and alternatively from about 10 to about 30. Non-limiting examples of classes of suitable bases include, but are not limited to ammonium salts, amines, hydroxides, metasilicates, and mixtures thereof. Non-limiting examples of suitable bases include sodium hydroxide, potassium hydroxide, sodium metasilicate, ammonium hydroxide, ethanolamine, aminomethylpropanol, ammonium carbonate, and mixtures thereof.

The third composition further may comprise from about 0.1% to about 10%, and alternatively from about 0.5% to about 5%, of at least one organic or inorganic salt. Examples of organic salts include, but are not limited to, salts formed by reacting at least one anion chosen from phosphates, borates, silicates, bicarbonates, carbonates, chlorates, nitrates, halides (including, but not limited to, chlorides), and/or sulfonates, with at least one cation chosen from potassium, sodium, strontium, cadmium, calcium, ammonium (such as tetraalkylammonium and arylammonium), phosphonium, barium, lithium, and/or magnesium. Non-limiting examples of suitable organic salts include sodium monobutyl and dibutyl phosphates and sodium monoethyl and diethyl phosphates. In one embodiment, the salt comprises an inorganic cation. In one embodiment, the inorganic cation is a multivalent cation selected from the group consisting of magnesium, calcium, strontium, barium, copper, zinc, iron, nickel, cobalt, manganese, aluminum, silver, lanthanum, and complexes and mixtures thereof.

### Dermatologically Acceptable Carrier

The compositions of the present invention may comprise from about 60% to about 99.9%, alternatively from about 70% to about 95%, and alternatively from about 80% to about 90%, of a dermatologically acceptable carrier. Carriers suitable for use with the composition(s) of the present invention include, for example, those used in the formulation of hair sprays, mousses, tonics, gels, and leave-on conditioners. The carrier may comprise water; organic oils; silicones such as volatile silicones, amino or non-amino silicone gums or oils, and mixtures thereof; mineral oils; plant oils such as olive oil, castor oil, rapeseed oil, coconut oil, wheatgerm oil, sweet almond oil, avocado oil, macadamia oil, apricot oil, safflower oil, candlenut oil, false flax oil, tamanu oil, lemon oil and mixtures thereof; waxes; and organic compounds such as C₂-C₁₀ alkanes, acetone, methyl ethyl ketone, volatile organic C₁-C₁₂ alcohols, esters of C₁-C₂₀ acids and of C₁-C₈ alcohols such as methyl acetate, butyl acetate, ethyl acetate, and isopropyl myristate, dimethoxyethane, diethoxyethane, C₁₀-C₃₀ fatty alcohols such as lauryl alcohol, cetyl alcohol, stearyl alcohol, and behenyl alcohol; C₁₀-C₃₀ fatty acids such as lauric acid and stearic acid; C₁₀-C₃₀ fatty amides such as lauric diethanolamide; C₁₀-C₃₀ fatty alkyl esters such as C₁₀-C₃₀ fatty alkyl benzoates; hydroxypropylcellulose, and mixtures thereof. In one embodiment, the carrier comprises water, fatty alcohols, volatile organic alcohols, and mixtures thereof

### Finishing Composition

The composition(s) of the present invention may further comprise a finishing composition including, but not limited, any form of product comprising an oxidizer such as hydrogen peroxide (about 0.1%-about 3% by weight) and buffered at low pH that is used to neutralize reduction and alkaline effects in the hair.

### Optional Components

The composition(s) of the present invention may further comprise one or more optional components known or otherwise effective for use in hair care or personal care products, provided that the optional components are physically and chemically compatible with the essential components described herein, or do not otherwise unduly impair product stability, aesthetics, or performance. Non-limiting examples of such optional components are disclosed in International Cosmetic Ingredient Dictionary, Ninth Edition, 2002, and CTFA Cosmetic Ingredient Handbook, Tenth Edition, 2004, both of which are incorporated by reference herein in their entirety. Some non-limiting examples of such optional components are disclosed below, and include plasticizers, surfactants (which may be anionic, cationic, amphoteric or nonionic), neutralizing agents, propellants, hair conditioning agents (e.g., silicone fluids, fatty esters, fatty alcohols, long chain hydrocarbons, cationic surfactants, etc.), emollients, lubricants and penetrants such as various lanolin compounds, vitamins, proteins, preservatives, dyes, tints, bleaches, reducing agents and other colorants, sunscreens, thickening agents (e.g., polymeric thickeners, such as xanthan gum), physiologically active compounds for treating the hair or skin (e.g., anti-dandruff actives, hair growth actives), non-polymeric thickeners including clays, and perfume.

The composition(s) of the present invention further may comprise from about 0.1% to about 10%, and alternatively from about 0.2% to about 5.0%, of a thickening agent to help provide the desired viscosity to the composition(s). Non-limiting examples of suitable optional gelling agents include crosslinked carboxylic acid polymers; unneutralized crosslinked carboxylic acid polymers; unneutralized modified crosslinked carboxylic acid polymers; crosslinked ethylene/maleic anhydride copolymers; unneutralized crosslinked ethylene/maleic anhydride copolymers (e.g., EMA 81 commercially available from Monsanto); unneutralized crosslinked allyl ether/acrylate copolymers (e.g., Salcare™ SC90 commercially available from Allied Colloids); unneutralized crosslinked copolymers of sodium polyacrylate, mineral oil, and PEC-1 trideceth-6 (e.g., Salcare™ SC91 commercially available from Allied Colloids); unneutralized crosslinked copolymers of methyl vinyl ether and maleic anhydride (e.g., Stabileze™ QM-PVM/MA copolymer commercially available from International Specialty Products), hydrophobically modified nonionic cellulose polymers; hydrophobically modified ethoxylate urethane polymers (e.g., Ucare™ Polyphobe Series of alkali swellable polymers commercially available from Union Carbide); and combinations thereof. In this context, the term “unneutralized” means that the optional polymer and copolymer gelling agent materials contain unneutralized acid monomers. Preferred gelling agents include water-soluble unneutralized crosslinked ethylene/maleic anhydride copolymers, water-soluble unneutralized crosslinked carboxylic acid polymers, water-soluble hydrophobically modified nonionic cellulose polymers and surfactant/fatty alcohol gel networks such as those suitable for use in hair conditioning products.

### III. METHODS OF USE

The present invention further describes methods of using the composition(s) of the present invention to chemically modify the internal portion of a hair shaft. The method comprises the step of applying to hair a moisture stabilization composition and an initiator as described herein. The initiator may form part of a second composition. The composition(s) may be applied by a variety of means, including with the fingers, hands, with an implement and/or with an energy delivery device. The composition(s) may be applied to wet hair or to dry hair. The amount applied will vary, and will depend upon the thickness and length of the hair, and the desired effect. In one embodiment, the composition(s) may be applied to substantially all of the hair, and alternatively to a portion of the hair. In one embodiment, the composition(s) may be applied to a portion of the hair that is proximal to the scalp, for example, from about 0 cm to about 10 cm, and alternatively from about 0 cm to about 5 cm proximal to the scalp. This may be desirable, for example, to “touch up” the roots of newly grown hair between treatments.

In one embodiment, the moisture stabilization composition comprising the monomer, and the second composition comprising the initiator are mixed prior to application to the hair, and applied to the hair as a single composition. For example, the first and second compositions may be mixed together one minute or less prior to application to the hair. Optionally, heat may be applied to the hair after applying the mixture. Alternatively, the moisture stabilization composition comprising the monomer is applied to the hair prior to application of the second composition comprising the initiator, without prior mixing. Alternatively, the second composition comprising the initiator is applied to the hair prior to application of the moisture stabilization composition comprising the monomer. The time interval between application of the moisture stabilization and the second composition may vary, and may comprise from about 5 minutes to about 60 minutes, alternatively from about 15 minutes to about 45 minutes, and alternatively may be about 30 minutes. Optionally, heat may be applied to the hair for all or for a portion of the time period between application of the compositions.

A suitable initiator also may include light energy. Therefore, in one embodiment, the moisture stabilization composition may be applied to the hair, and the hair exposed to light energy. One non-limiting example includes allowing the composition to remain on the hair under a light source for about 5 minutes to about 30 minutes.

The method further may comprise the step of applying a third, or pH adjusting composition to the hair. The third composition may be mixed with the first and/or second composition, such that the final pH of the mixture of compositions is greater than 7.0, alternatively from 7.0 to about 12.0, alternatively from about 8.0 to about 12.0, and alternatively is from about 9.0 to about 11.0.

The method further may comprise the step of applying a fourth, or reducing, composition to the hair. In one embodiment, the reducing composition is applied prior to one or more compositions described herein and is allowed to remain on the hair from about 5 minutes to about 60 minutes, and in another embodiment the reducing composition remains on the hair for about 15 minutes, after which time the composition is substantially removed from the hair, for example by rinsing with water. Optionally, heat may be applied to the hair for all or for a portion of the time during which the composition remains on the hair. The fourth, or reducing, composition, comprises from about 1% to about 12%, alternatively from about 4% to about 10%, and alternatively from about 8% to about 10%, of a reducing agent. Examples of suitable reducing agents include, but are not limited to, sodium thioglycolate, anhydrous sodium thiosulfate, powdered sodium metabisulfite, thiourea, ammonium sulfite, thioglycolic acid, thiolactic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formosulfoxylate, isooctyl thioglycolate, dl-cysteine, monoethanolamine thioglycolate, phosphines, thioglycolic acid, dithiothreitol, dihydrolipoic acid, N-(3′hydroxypropyl)-2-mercaptoacetamide, N[Bis-(2-methoxyethyl)]-2-mercaptopropionamide2,6-Dimethyl-4-mercaptoacetylmorpholin, N-(3-methoxypropyl)-2-mercaptopropionamide, N-Tetrahydrofurfuryl-2-mercaptopropionamide, 2-Mercapto-(3-Hydroxy-N,N-pentamethylen)-propionamide, and mixtures thereof.

The method further may comprise a finishing step of applying a finishing composition which can be a neutralizing, and/or oxidizing composition to the hair. In one embodiment, the finishing composition is applied after one or more of the compositions described herein are substantially removed from the hair, for example by rinsing with water and is allowed to remain on the hair from about 1 minute to about 5 minutes.

After treatment with one or more compositions of the present invention, the hair may exhibit one or more benefits in addition to moisture stabilization, including but not limited to increased shape retention and/or durability, increased appearance of volume, enhanced shine, better color retention, and easier hair manageability. Examples include all day hold of style, excellent curl definition, increased body and/or fullness, the ability to curl straight hair, and/or the ability to straighten curly hair.

### IV. KIT

The present invention further describes a kit comprising a moisture stabilization composition and optionally a second composition as described herein. The first and the second composition may be packaged in separate containers within the kit, and alternatively may be packaged in a single container which is capable of preventing admixing of the two compositions. The packaging may be of a size suitable for a single application, or unit dose, of the first and/or second composition. The kit may comprise a number of unit doses suitable for an indicated treatment regimen.

The kit further may comprise at least one additional composition selected from the group consisting of a pH adjustor, a reducing composition, and combinations thereof. In one embodiment, the pH adjustor has a reserve alkalinity as described herein and packaged in a container having a volume, such that when the contents of the container are mixed with the first and/or the second composition, the pH of the resulting mixture of compositions is greater than 7.0.

The kit further may comprise at least one additional component selected from the group consisting of a shampoo, a conditioner, a neutralizer, a colorant, a styling aid such as a gel, a mousse, a pomade, etc., an implement, an energy delivery device, instructions for complying with a treatment regimen, and combinations thereof. Examples of energy delivery devices include, but are not limited to light sources, including UV, visible light and infrared light, temperature change elements, hair dryers, heaters such as irons and heated curlers, ultrasonic devices, etc.

The kit further may comprise instructions for complying with a hair treatment regimen. The treatment regimen may comprise one or methods of use described herein, and may be directed toward treatment by a professional stylist or toward treatment by a consumer who is not a professionally-trained stylist.

### V. ARTICLE OF COMMERCE

The present invention further describes an article of commerce comprising a moisture stabilization composition and/or a second composition, as described herein, and a communication pertaining to the compositions. The communication may be printed material attached directly or indirectly to packaging, for example to a kit that contains the compositions. Alternatively, the communication may be placed directly or indirectly near at least one composition. Alternatively, the communication may be an electronic or a broadcast message that is associated with the applicator and/or the composition. The communication may comprise images comparing the appearance of a person prior to use of the compositions to the appearance of the same person after use of the composition.

### VI. EXAMPLES

The following describes one non-limiting example of a method of using the compositions of the present invention to maintain and/or stabilize the moisture level in the hair shaft:

Begin with dry hair. If the hair is washed first, make sure hair is thoroughly dried, and comb the hair sufficiently to detangle. Mix together by stirring or vigorously shaking, for about 30 seconds, about 50 ml of a moisture stabilization composition (Composition I—described below), about 1.0 ml of a second composition (Composition II—described below), and about 49 ml of a pH adjusting composition (Composition III—described below). Apply a suitable amount, as described above in this example, of the mixture to the hair. Gently massage the mixture into the hair, avoiding contact of the composition with the skin and/or scalp and eyes. Allow the composition to remain on the hair, uncovered, for about 30 minutes at 35° C.±5° C. Gently wash the hair (preferably with clarifying shampoo) and rinse thoroughly with water. Optionally, a conditioner may be applied to the hair. The hair may further be dried and styled as desired.

Composition I

| | | Example 1 | Example 2 | Example 3 | Example 4 | Example 5 |
|---|---|---|---|---|---|---|
| | | wt. % | wt. % | wt. % | wt. % | wt. % |
| | Ingredients: | | | | | |
| 1 | Purified Water | qs | qs | qs | qs | qs |
| 2 | Hydroxypropylmethyl | 0.1 | 0.1 | 0.2 | 0.2 | 0.3 |
| | cellulose | | | | | |
| 3 | Polyquaterium-10 | 1.0 | — | 1.0 | — | 1.2 |
| 4 | Cremophor EL | 0.2 | — | 0.2 | 0.2 | 0.2 |
| 5 | Tetrasodium EDTA | 0.05 | 0.05 | 0.05 | 0.05 | 0.05 |
| 6 | Phenoxyethanol | 0.25 | 0.25 | 0.25 | 0.25 | 0.25 |
| 7 | Sodium Benzoate | 0.125 | 0.125 | 0.125 | 0.125 | 0.125 |
| 8 | Ascrobic Acid | 0.05 | 0.05 | 0.05 | 0.05 | 0.05 |
| 9 | 1,2-Hexanediol | — | 2 | 2 | — | — |
| 10 | Isopropyl alcohol | 10 | — | 10 | — | — |
| 11 | Genepol C-100 | — | 0.2 | | 0.2 | 0.2 |
| | Monomer Actives: | | | | | |
| 12 | Hydroxymethyl Diacetone | 12.8 | 12 | 8 | — | 8 |
| | Acrylamide | | | | | |
| 13 | Butoxyethylacrylate | 3.2 | — | 7 | — | 2 |
| 14 | 2-Acrylamidoglycolic Acid | | | | 16 | 5.5 |
| | Monohydrate | | | | | |
| 15 | 3-(Acryloyloxy)-2-hydroxy- | — | 1.5 | 1 | — | 0.5 |
| | propyl methacrylate | | | | | |
| | Fragrance: | | | | | |
| 16 | Mimo | — | 0.25 | 0.25 | 0.25 | 0.25 |

Mix the ingredients as follows, with continuous stirring. Add water to a suitable mixing vessel. Heat to about 80° C. Add ingredients 2-11 and stir until dissolved and allow temperature to again reach about 80° C. Maintain temperature at about 80° C. for about 10 minutes. Cool to below 30° C. Add ingredients 12-16 and continue to stir until dissolved.

Composition II

| | | Example 1 | Example 2 | Example 3 | Example 4 | Example 5 |
|---|---|---|---|---|---|---|
| | Ingredients: | wt. % | Wt. % | Wt. % | Wt. % | wt. % |
| 16 | Sodium Persulfate | 100 | — | 90 | 10 | 50 |
| 17 | Hydroxymethanesulfinic | — | 100 | — | 90 | 50 |
| | acid monosodium salt | | | | | |
| 18 | Copper (III) Nitrate | — | — | 10 | — | — |

Combine ingredients. Mixture may be neat. Composition II may not be a solution but can be packaged as a dry powder or crystalline substance.

Composition III

| | | Example 1 | Example 2 | Example 3 | Example 4 | Example 5 |
|---|---|---|---|---|---|---|
| | Ingredients: | wt. % | wt. % | wt. % | wt. % | wt. % |
| 19 | Purified Water | qs | qs | qs | qs | qs |
| 20 | Ammonium | 8 | — | — | — | 10 |
| | Hydroxide (5N | | | | | |
| | standard) | | | | | |
| 21 | Sodium Hydroxide | — | 10 | — | — | — |
| | (1N standard) | | | | | |
| 22 | Sodium Metasilicate | — | — | 10 | — | — |
| 23 | Strontium Chloride | — | 5 | 5 | — | — |

Add water to a suitable mixing vessel. Add ingredients 20-23 and continue to stir until dissolved.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for maintaining moisture in the hair shaft comprising the steps of:
chemically modifying the internal region of a hair shaft by applying to the hair a composition comprising:
a) a copolymer comprising
i. at least one hydrophilic monomer, wherein the hydrophilic monomer is selected from the group consisting of sulfopropyl(meth)acrylate compound, 2-acrylamidodiglycolic acid, hydroxymethyl diacetone acrylamide, (3-acrylamidopropyl)trimethyl ammonium salt, 2-aminoethyl methacrylate and mixtures thereof; and
ii. at least one hydrophobic monomer, wherein the hydrophobic monomer is selected from the group consisting of 2-ethoxyethyl acrylate, 2-butoxyethyl acrylate, alpha methylene lactone and mixtures thereof; and
b) a dermatologically acceptable carrier.

2. The method of claim 1, comprising from about 20% to about 99% hydrophilic monomer.

3. The method of claim 2, comprising from about 25% to about 75% hydrophilic monomer.

4. The method of claim 1, comprising from about 1% to about 80% hydrophobic monomer.

5. The method of claim 4, comprising from about 5% to about 20% hydrophobic monomer.

6. The method of claim 1, wherein the hydrophilic monomer is hydroxymethyl diacetone acrylamide.

7. The method of claim 1, wherein the hydrophobic monomer is butoxyethylacrylate.

8. The method of claim 1, wherein the hydrophobic monomer is hydroxymethyl diacetone acrylamide and the hydrophilic monomer is butoxyethylacrylate.

9. The method of claim 1, wherein the hydrophilic and the hydrophobic monomers have neutralizable groups such as carboxylates or quaternium amines.

10. The method of claim 1, wherein the composition further comprises an initiator selected from the group consisting of peroxodisulfates, peroxides, peracids, percarbonate, phosphates, manganates, borates, bis-alkyamidines, sulfites, peroxyesters, bis-cyanocarboxylic acids, alpha-amino acetic acids, sodium peroxydisulfate, 2,2′-azobis(2-methylpropionamidine)dihydrochloride, 2,2′-azobisisobutyronitrile, benzoyl peroxide, peracetic acid, ammonium cerium(IV) nitrate, hydroxymethanesulfinic acid, and mixtures thereof.

11. The method of claim 1, wherein the composition is mixed prior to application to the hair.

12. The method of claim 1, further comprising the step of applying heat to the hair.

13. The method of claim 1, wherein the composition is applied to a portion of the hair proximal to the scalp.

14. The method of claim 1, wherein the composition further comprises from about 0.1% to about 20% of at least one additional compound selected from the group consisting of an ethylenic monomer, a crosslinker, and mixtures thereof, wherein the additional compound has a molecular weight of less than 500 g/mole.

15. The method of claim 14, wherein the composition comprises a crosslinker.

16. The method of claim 15, wherein the crosslinker is selected from the group consisting of 1,4-bisacryloylpiperazine, methylenebisacrylamide, ethylenebisacrylamide, divinylbenzene, poly-ethyleneglycol di(meth)acrylate, ethylene glycol di(meth)acrylate, 1,3-butanediol di(meth)acrylate, 1,4-butanediol di(meth)acrylate, neopentyl glycol di(meth)acrylate, Bis[2-(methacryloyloxy)ethyl]phosphate, N,N′-bis(acryloyl)cystamine, N N-Diallylacrylamide, triallyl cyanurate, 3-(Acryloyloxy)-2-hydroxypropyl methacrylate and mixtures thereof.

17. The method of claim 1, wherein the composition further comprises from about 0.1% to about 1% of a catalyst.

18. The method of claim 1, further comprising the step of determining the humidity of the environmental conditions.

19. The method of claim 18, further comprising modifying the composition for dry environmental conditions.

20. The method of claim 19, whereas the copolymer comprises from about 1% to about 10% hydrophobic monomer and from about 90% to about 99% hydrophilic monomer

21. The method of claim 18, further comprising modifying the composition for humid conditions.

22. The method of claim 21, wherein the copolymer comprises from about 40% to about 60% hydrophobic monomer and about 40% to about 60% hydrophilic monomer.

23. The method of claim 1, further comprising the step of adding a composition to the hair selected from the group consisting of a neutralizing composition, an oxidizing composition, and a combination thereof.

24. A hair care composition comprising a copolymer comprising:
from about 20% to about 99% hydrophilic monomer, wherein the hydrophilic monomer is hydroxymethyl diacetone acrylamide, and
from about 1% to about 80% of a hydrophobic monomer, wherein the monomer is butoxyethylacrylate; and
a dermatologically acceptable carrier.
